(19) United States
(12) Patent Application Publication
(10) Pub. No.: US 2008/0311166 A1
(43) Pub. Date: Dec. 18, 2008

(54) COOLING TOWEL

(76) Inventor: ALLAN H. WIMER, Key West, FL (US)

Correspondence Address: AKERMAN SENTERFITT, P.O. BOX 3188, WEST PALM BEACH, FL, 33402-3188, US

(21) Appl. No.: 11/762,947

(22) Filed: Jun. 14, 2007

(51) Int. Cl. A61K 36/886

(52) U.S. Cl. 424/402; 424/744; 442/123

(57) ABSTRACT

Absorbent cloths (cooling towels) impregnated with a formulation including purified water, denatured alcohol, Aloe vera, menthol, and at least one scented essential oil, have been developed for quickly and conveniently cooling the skin. A typical cooling towel as described herein is made from 100% cotton terry cloth, made of all-natural ingredients that are environmentally friendly, and is convenient and easy to use. The cooling towels described herein can be used to cool a user's skin in a variety of settings and applications (e.g. while playing golf, for relief from menopausal hot flashes, etc.). A cooling towel is capable of providing instant cooling relief to the user without refrigerating or hydrating the cooling towel. Placing a cooling towel around the neck, for example, aids in lowering body temperature by helping dissipate body heat to provide immediate relief. For even better cooling results, a cooling towel as described herein can be refrigerated or frozen prior to use to enhance the cooling effect. A cooling towel as described herein can be sealed in a package (e.g., plastic) such that when removed from the package, the cooling towel quickly becomes cool and remains cool for one to four hours. Cooling towels can be sized to conveniently fit inside a purse, pocket, glove box, lunch box, cooler, etc.

### STATEMENT AS TO FEDERALLY SPONSORED RESEARCH

Not applicable.

### FIELD OF THE INVENTION

The invention relates generally to the fields of textiles, athletics, and personal hygiene. More particularly, the invention relates to absorbent cloths for cooling a user's body.

### BACKGROUND

There are many occasions during or after which a means for cooling hot (and possibly sweaty) skin is highly desirable. For example, after hiking, jogging, fishing, playing tennis, roller blading, golfing, cycling, dancing, working out, or while sitting in a hot stadium, a means for quickly cooling and refreshing oneself would be particularly useful. For athletes in particular, a quick cool-down can be vital for preventing heat stroke and for energizing the body while performing. A number of devices for cooling the skin are commercially available. For example, the Sammy Cool 'N Dry (Super Cool Products, Inc., Elmhurst, Ill.) is made of a PVA polymer material which is highly absorbent, one side having a waffle texture for fast cooling. To use this towel, a user wets the towel with water, rings it out, and the capillary action of the micropores allows the towel to become about 20 degrees cooler than the existing air temperature. Another device for cooling the skin is the Chilly Pad (frogg toggs® Arab, Ala.). This towel is made from an evaporative material that retains water while remaining dry to the touch. When wet, the towel becomes considerably cooler than the outside air, thereby providing cooling relief to the user. Hima Ice Towel Corp. (Westlake, Ohio) offers a prepackaged cotton towel soaked with essential plant oils that needs to be frozen for 12 hours prior to use.

These commercially available devices, however, require freezing for several hours prior to use or the addition of water to achieve any cooling effect. A towel capable of providing instant cooling relief without the addition of water or refrigeration or freezing would be extremely convenient and would satisfy a great need.

### SUMMARY

The invention relates to the development of cooling towels, absorbent cloths impregnated with a formulation including purified water, denatured alcohol, Aloe vera, menthol, and at least one scented essential oil for quickly and conveniently cooling the skin of a user (e.g., a human). A typical cooling towel as described herein is made from 100% cotton terry cloth, made of all-natural ingredients that are environmentally friendly, and is convenient and easy to use. The cooling towels described herein can be used to cool a user's skin in a variety of settings and applications (e.g. during any sports activity such as golf). A cooling towel is capable of providing instant cooling relief to the user in any application where heat relief is desired or needed (e.g., a medical condition such as menopause). Placing a cooling towel around the neck, for example, aids in lowering body temperature by helping dissipate body heat to provide immediate relief. For even better cooling results, a cooling towel as described herein can be refrigerated or frozen prior to use to enhance the cooling effect. A cooling towel as described herein can be sealed in a package (e.g., plastic) such that when removed from the package and exposed to air, the cooling towel quickly becomes cool (e.g., within seconds) and remains cool for one to four hours (e.g., 1, 2, 3, 4 hours) depending on the climate in which the cooling towel is used, the cooling effect lasting longer in a humid climate than in an arid climate. Cooling towels can be sized to conveniently fit inside a purse, pocket, glove box, lunch box, cooler, etc.

Accordingly, the invention features a formulation for cooling an external surface of a mammalian (e.g., human) body, the formulation including: purified water at a concentration of about 67% to about 97% by volume; denatured alcohol at a concentration of about 3.2% to about 26.7% by volume; Aloe vera at a concentration of about 0.3% to about 4.2% by volume; menthol at a concentration of about 0.3% to about 4.2% by volume; and at least one scented essential oil at a concentration of about 0.3% to about 3.3% by volume.

Also included within the invention is an absorbent cloth that when applied to an external surface of a mammalian body results in cooling of the external surface. The absorbent cloth includes an absorbent cloth impregnated with a formulation including: purified water; denatured alcohol; Aloe vera; menthol; and at least one scented essential oil. The purified water can be at a concentration of about 67% to about 97% by volume, the denatured alcohol can be at a concentration of about 3.2% to about 26.7% by volume, the Aloe vera can be at a concentration of about 0.3% to about 4.2% by volume, the menthol can be at a concentration of about 0.3% to about 4.2% by volume, and the essential oil can be at a concentration of about 0.3% to about 3.3% by volume. The absorbent cloth can further include a second essential oil. The denatured alcohol can be ethyl alcohol, the Aloe vera can be Aloe vera extract, the scented essential oil can be citrus essential oil, and the absorbent cloth can be terry cloth. The absorbent cloth can have a width in the range of about 6 inches to about 15 inches and a height in the range of about 9 inches to about 30 inches. The absorbent cloth can have a width of about 12 inches and a height of about 27 inches. An absorbent cloth can also have a width of about 12 inches and a height of about 12 inches. The absorbent cloth is capable of cooling the external surface for a time period up to about four hours.

In another aspect, the invention features a method of cooling at least one external surface of a mammalian body. The method includes the steps of: providing an absorbent cloth impregnated with a formulation including purified water, denatured alcohol, Aloe vera, menthol, and at least one scented essential oil; and applying the absorbent cloth to the at least one external surface, wherein when applied to the at least one external surface, the absorbent cloth results in cooling of the at least one external surface. In the method, the formulation can further include a second essential oil, the denatured alcohol can be ethyl alcohol, the Aloe vera can be Aloe vera extract, the scented essential oil can be citrus essential oil, and the absorbent cloth can be terry cloth. The absorbent cloth can have a width in the range of about 6 inches to about 15 inches and a height in the range of about 9 inches to about 30 inches. The width can be about 12 inches and the height about 27 inches. The width can be about 12 inches and the height about 12 inches. The cooling of the at least one external surface can occur for a time period between two and three hours.

Unless otherwise defined, all technical terms used herein have the same meaning as commonly understood by one of ordinary skill in the art to which this invention belongs.

By the phrase “terry cloth” is meant a pile fabric, usually of cotton, with uncut loops.

As used herein, a “cooling towel” means an absorbent cloth impregnated with purified water, denatured alcohol, Aloe vera, menthol, and at least one scented essential oil.

By the term “essential oil” is meant any volatile oil obtained from a plant and having the characteristic odor or flavor of the plant from which it is obtained. For example, peppermint essential oil is obtained from a peppermint plant. “Essential oils” as used herein may include one or more volatile oils each obtained from a different plant.

As used herein, the term “menthol” means a compound recognized as menthol as a chemical compound or an equivalent of menthol as a chemical compound by a person of ordinary skill in the art. Menthol is also referred to as “hexahydrothymol” and “peppermint camphor.” The term “menthol” is intended to cover all isomers of 5-methyl-2-(1-methylethyl)-cyclohexanol in an levorotatory (l), dextrorotatory (d) or racemic (dl) form. Isomers of menthol include (−)-menthol, (+)-menthol, (−)-isomenthol, (+)-isomenthol, (−)-neomenthol and (+)-neomenthol and any mixture thereof. Equivalents of menthol include menthol analogues such as cis-p-menth-2-en-ol, and salts or esters of menthol as a chemical compound and menthol extracts derived from plants of the mint family, for example, the menthol extract of peppermint oil. The menthol compound can be derived from natural sources or synthetically made. Menthol USP as identified in the United States Pharmacopeia (USP) may be levorotatory, from natural or synthetic sources, or racemic.

By the term “Aloe vera” is meant a compound, extract, or other preparation obtained from an Aloe plant in the genus Aloe, including the species A. barbadensis and A. arboresens. The term as used herein encompasses any biologically active compound isolated from Aloe vera, including free and conjugated anthraquinones, chromone glycosides, polysaccharides, and alpha hydroxy acids.

As used herein, “purified water” means water that has been treated to remove impurities.

By the term “denatured alcohol” is meant an alcohol that has been denatured. Denatured alcohol can be, for example, ethanol to which a denaturant has been added, the denaturant rendering it unfit for drinking.

Although compositions, formulations, and methods similar or equivalent to those described herein can be used in the practice or testing of the present invention, suitable methods and materials are described below. All publications, patent applications, and patents mentioned herein are incorporated by reference in their entirety. In the case of conflict, the present specification, including definitions, will control. The particular embodiments discussed below are illustrative only and not intended to be limiting.

### DETAILED DESCRIPTION

A cooling towel for cooling the skin as described herein is an absorbent cloth impregnated with a formulation including purified water, denatured alcohol, Aloe vera, menthol, and at least one scented essential oil. A pre-moistened cooling towed is typically individually wrapped in a hygienically sealed package. When removed from the package, the pre-moistened towel is moist and cool and ready for use. When applied to an external surface of a user's body (e.g., neck, arm, forehead), the cooling towel results in cooling of the body surface. Cooling towels require neither refrigeration nor hydration to provide the cooling sensation. The cooling sensation provided by a cooling towel can last between about one hour and about four hours depending on the temperature and humidity of the climate in which a cooling towel is being used. Generally, the cooling effect lasts longer in a hot and humid climate than it lasts in an arid environment.

An absorbent cloth as described herein is 100% cotton terry cloth in a typical embodiment. However, any suitable absorbent material can be used, e.g., linen, paper, polyester, rayon, denim, and any other synthetic material. A cooling towel is typically disposable (i.e., intended for a single use) and biodegradable. A cooling towel can be any shape, and any suitable size. A cooling towel in the shape of a rectangle or square, for example, can have a width from about 3 to about 18 (e.g., 3, 4, 5, 6, 7, 8, 9, 10, 11, 12, 13, 14, 15, 16, 17, 18 etc.) inches and a height of about 6 to about 42 (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, etc.) inches. Generally, the width of a cooling towel is about 6 to about 15 inches, and the height is about 9 to about 30 inches. A cooling towel sized suitably for many applications is approximately 12 inches by 12 inches. The size of the cooling towel may depend on the application for use of the cooling towel. For example, for use by golfers, a cooling towel as described herein can be approximately 12 inches by 27 inches, sized comparably to a golf towel. However, any suitable dimensions can be adapted to a cooling towel as described herein, e.g., 6 inches by 6 inches, 8 inches by 8 inches, 10 inches by 10 inches, 12 inches by 20 inches, 12 inches by 36 inches, 12 inches by 38 inches, 12 inches by 42 inches, etc.

Absorbent cloths as described herein are impregnated with a formulation including purified water, denatured alcohol, Aloe vera, menthol, and at least one scented essential oil. The combination of denatured alcohol, purified water, Aloe vera and menthol provides a cool sensation (e.g., approximately 20-25 degrees F. below body temperature) by enhancing the body's natural cooling process of evaporative cooling by perspiration. Including at least one plant essential oil, such as a citrus oil, in the formulation results in the cooling towel having a citrus fragrance that is refreshing and pleasing to the nose, while Aloe vera soothes and moisturizes the skin. In one example of a formulation, the formulation includes water that has been purified by distillation, ethyl alcohol, Aloe vera extract, menthol, and citrus essential oil (see Example 1). Although the purified water, denatured alcohol, Aloe vera, menthol, and at least one scented essential oil are natural in a typical embodiment (i.e., having undergone little or no processing and containing no chemical, artificial, or synthetic additives), in other embodiments, one or more of these components may be synthesized.

In the formulations, purified water acts as a natural moisturizer and is typically included at a concentration of about 67% to about 97% (e.g., 66%, 67%, 70%, 75%, 80%, 85%, 90%, 94% 97%) by volume. Purified water from any suitable source can be used, e.g., water that has been obtained from a spring, an ocean (or other body of water), a municipal water system, or rain. Methods for purifying water are well known, and water for use in formulations described herein can be purified by any suitable purification process, including distillation, filtration, boiling, and reverse osmosis.

Any suitable denatured alcohol can be included in the formulations described herein. For example, denatured ethyl alcohol can be used. Another example of a denatured alcohol that can be used in the formulations described herein is SD alcohol 40-B. SD alcohol 40-B is ethanol that has been denatured by the addition of denatonium benzoate. Examples of additional alcohols that can be denatured and used in compositions and methods of the invention include methanol and isopropanol. Substances that can be used to denature alcohol include but are not limited to Alpha terpineol, Anise oil, Benzaldehyde, denatonium benzoate, Camphor, Coal tar, Eucalyptol, Lavender, Menthol, Phenol, Storax, and Thymol. See part 21 of title 27 of the Code of Federal Regulations (27 CFR §21.65) for methods of denaturing alcohol. Denatured alcohol is typically included at a concentration of about 3.2% to about 26.7% (e.g., 3.2%, 4.4%, 10%, 15%, 20%, 25%, 26.7%, 27%) by volume in a formulation as described herein. After a cooling towel is applied to the skin, the denatured alcohol evaporates from the skin, thereby removing heat from the skin and transferring it to the surrounding air.

In the formulations described herein, Aloe vera provides natural cooling sensation and skin moisturizing properties. Aloe vera is typically included at a concentration of about 0.3% to about 4.2% (e.g., 0.3%, 0.5%, 1.0%, 1.5%, 2.0%, 2.5%, 3.0%, 3.5%, 4.0%, 4.2%) by volume in a formulation for impregnating an absorbent cloth. Any suitable Aloe vera can be used in the formulations described herein, e.g., Aloe vera from A. barbadensis Mill, A. vulgaris Lam, etc. In a typical embodiment, 100% natural Aloe vera extract from A. barbadensis Mill and A. vulgaris Lam is used (Thai China Flavours and Fragrances Industry Co., Ltd. Bangkok, Thailand).

Any suitable menthol can be used in formulations for impregnating an absorbent cloth. For example, 100% natural, organic menthol obtained from peppermint essential oil can be used. Menthol typically is included at a concentration of about 0.3% to about 4.2% (e.g., 0.3%, 0.5%, 1.0%, 1.5%, 2.0%, 2.5%, 3.0%, 4.0%, 4.2%, 4.5%) by volume in a formulation as described herein. Menthol is a physiological stimulus to human skin receptors that can be activated by cool temperatures, thus providing a cooling sensation. Menthol is a denaturant of ethyl alcohol, and can also be used as a denaturant in formulations described herein. When used as a denaturant, methanol is included at a concentration of about 0.3% to about 3.9%.

Any suitable scented essential oil can be included in a formulation as described herein. A non-exhaustive list of essential oils includes Frankincense, Bergamot, Lime, Lemon, Mandarin, Lemongrass, Citronella, Eucalyptus, Lavender, Vetiver, Sandalwood, Clary Sage, Rosemary, Pine, Aniseed, Basil, Spearmint, Peppermint, Melissa, Cajuput, Chamomile, Immortelle, Myrrh, Grapefruit, Petitgrain, and Angelica. The choice of essential oil can be based on scent, or on other properties provided by a particular essential oil. For example, Lavender essential oil is known to have calming, relaxing properties. In the formulation of Example 1, 100% natural citrus essential oil (obtained from plants flowering plants of the Rutcaeae family originating in tropical and subtropical southeast Asia) was included to provide a fresh, clean, scent. More than one scented essential oil can be included, e.g., a combination of Lavender and Clary Sage. In some embodiments, one or more synthetic scented oils can be added to a formulation for impregnating an absorbent cloth, e.g., Ocean Breeze commercially available from Thai China Flavours and Fragrances Industry Co., Ltd. Bangkok, Thailand. Ocean Breeze scented oil is only one example of a synthetic scented oil that can be used in compositions, formulations, and methods as described herein. Many synthetic scented oils are commercially available, and any suitable synthetic scented oil can be used. The at least one scented essential oil is typically included at a concentration of about 0.3% to about 3.3% (e.g., 0.3%, 0.5%, 1.0%, 1.5%, 2.0%, 2.5%, 3.0%, 3.3%, 3.5%) by volume in a formulation as described herein. In addition to the at least one scented essential oil, one or more essential oils can be added to the formulation for denaturing alcohol.

Cooling towels as described herein can be prepared by any suitable method. Typically, a formulation including purified water, denatured alcohol (e.g., ethanol that is added to a formulation and that becomes denatured by menthol and/or one or more essential oils), Aloe vera, menthol and at least one scented essential oil is first prepared (see, e.g., Example 1). An absorbent cloth to be impregnated with such a formulation is generally sterilized before being impregnated with the formulation. For example, an absorbent cloth can be sterilized one or more times by any suitable method (e.g., boiling). After the formulation and the absorbent cloth are prepared, the absorbent cloth is then contacted with a sufficient volume of formulation such that the absorbent cloth is impregnated with the formulation. The formulation can be sprayed onto the absorbent cloth, or it can be contacted with the absorbent cloth by other methods. For example, an absorbent cloth can be dipped into a vessel containing the formulation. Although an absorbent cloth is typically contacted with a formulation as described herein, an absorbent cloth can be contacted with one or more of purified water, denatured alcohol, Aloe vera, menthol and at least one scented essential oil individually (e.g., sequentially), or in one or more combinations thereof. In such a method, the purified water, denatured alcohol, aloe vera, menthol and at least one scented essential oil can be added to an absorbent cloth in any order to produce a cooling towel as described herein. The amount of formulation used to impregnate an absorbent cloth depends on a number of factors, including the size, thickness, and type of material of the absorbent cloth. An absorbent cloth should be sufficiently impregnated with formulation such that it provides a cooling effect, but not so impregnated with the formulation that formulation drips from the absorbent cloth while the user is holding or using the cooling towel. In a typical embodiment, a 12 by 12 inch terry cloth absorbent cloth is impregnated with about 35 grams of formulation. A 12 inch by 12 terry cloth absorbent cloth can be impregnated, however, with between about 26 and about 48 grams of formulation (e.g., 25, 30, 35, 40, 45, 50 grams). In another embodiment, a 12 inch by 27 inch terry cloth absorbent cloth is impregnated with about 90 grams of formulation, but can be impregnated with between about 85 grams and 110 grams of formulation (e.g., 85, 90, 95, 100, 105, 110 grams).

Absorbent cloths as described herein can come in a variety of colors, and can have a variety of textures. In some embodiments, a cooling towel may be embroidered with a logo or design.

After a cooling towel is made by one of the methods described herein, the pre-moistened cooling towel is typically individually wrapped in a hygienically sealed package (e.g., plastic). When removed from the package, the pre-moistened towel is moist and cool and ready for use. In a typical embodiment, one cooling towel is packaged per sealed package. In a similar embodiment, a plurality of sealed packages (each containing one cooling towel) can be packaged together as one unit (e.g., a “multi-pack”). In other embodiments, a plurality (e.g., 2, 3, 4, 5, 6, 7, 8, 9, 10, etc.) of cooling towels can be packaged in a single sealed package.

Cooling towels and methods of using cooling towels for cooling an external surface of a mammalian body as described herein may find use in a variety of activities or applications. As one example, cooling towels may find particular use by golfers, especially those playing golf in a hot climate. By placing a cooling towel as described herein on the neck, for example, a golfer can feel instant cooling relief from the heat. Placing a cooling towel around the neck aids in lowering the body temperature of the user by helping to dissipate body heat, thereby providing a cooling effect and relief. In another example, cooling towels may be used while or subsequent to biking, jogging, or playing tennis. In yet another example, a woman experiencing menopause may find a cooling towel particularly cooling and soothing. Because the cooling towel contains water and denatured alcohol, it can also be used as a cleansing towel. A cooling towel as described herein also has natural anti-microbial and antiseptic properties due to the presence of denatured alcohol and in some embodiments due to the citrus essential oil, and may be particularly useful to a user who is traveling or for use by children. If stored in a refrigerator or freezer prior to use, a cooling towel confers a greater cooling effect then if not pre-cooled.

### EXAMPLES

The present invention is further illustrated by the following specific examples. The examples are provided for illustration only and should not be construed as limiting the scope of the invention in any way.

### Example 1

### A First Embodiment of a Cooling Towel

In a first embodiment of a cooling towel for cooling the body, a 100% cotton natural terry cloth towel was impregnated with the following:

a) purified water at 94% by volume,

b) denatured ethyl alcohol at 4.4% by volume,

c) 100% Aloe vera extract (100% natural extract from A. barbadensis Mill and A. vulgaris Lam purchased from Thai China Flavours and Fragrances Industry Co., Ltd. Bangkok, Thailand) at 0.5% by volume,

d) menthol (100% natural organic compound made from peppermint essential oil) at 0.5% by volume, and

e) citrus essential oil (from flowering plants of the Rutcaeae family) at 0.6% by volume.

In this example, the denatured ethyl alcohol was prepared by adding natural essential oil denaturants to a grain-based alcohol. Components a) through e) were added to a vessel and mixed, resulting in a formulation for impregnating an absorbent cloth. The absorbent cloths to be impregnated were sterilized twice by boiling before being impregnated with the formulation. Each of the components in this example are natural and free of chemicals. Cooling towels made by this method were individually sealed in plastic packages such that when removed from the packages, the cooling towels became cool within a brief period of time from opening the packaging (i.e., several seconds) and remained cool for about four hours.

### Example 2

### A Second Embodiment of a Cooling Towel

A cooling towel to be used by menopausal women for relief from hot flashes is impregnated with a formulation including purified water (e.g., purified water at 94% by volume), denatured alcohol (e.g., denatured ethyl alcohol at 4.4% by volume), Aloe vera (e.g., 100% Aloe vera extract at 0.5% by volume), menthol (e.g., 0.5% by volume), and Clary Sage essential oil (e.g., 0.6% by volume). Both the Clary Sage fragrance and the coolness provided by the cooling towel provides relief from hot flashes. A cooling towel to be used by menopausal women for relief from hot flashes is prepared and used as described above.

### Example 3

### Additional Embodiments of a Cooling Towel

Additional embodiments of a cooling towel are encompassed by the invention. For example, an aromatherapy towel can be impregnated with a formulation including purified water, denatured alcohol, Aloe vera, menthol, and one or more essential oils providing therapeutic benefits. As another example, a sunscreen cooling towel can be impregnated with a formulation including purified water, denatured alcohol, Aloe vera, menthol, at least one essential oil, and at least one compound for protecting a user's skin from ultraviolet radiation (e.g., oxybenzone, zinc oxide, etc.). As yet another example, an insect repellent towel can be impregnated with a formulation including purified water, denatured alcohol, Aloe vera, menthol, at least one essential oil, and at least one compound for discouraging insects from landing on or climbing on a user's skin (e.g., N,N-diethyl-m-toluamide, p-menthane 3,8-diol, citronella oil, etc.). A fourth example is an anti-bacterial cooling towel impregnated with a formulation including purified water, denatured alcohol, Aloe vera, menthol, at least one essential oil, and at least one anti-septic reagent. In this example, the at least one anti-septic reagent can be an anti-septic solution such as Avagard™ (commercially available from 3M™, St. Paul, Minn.).

### OTHER EMBODIMENTS

Any improvement may be made in part or all of the components. All references, including publications, patent applications, and patents, cited herein are hereby incorporated by reference. In any listing of possible components, mixtures of possible components are contemplated unless expressly indicated otherwise. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A formulation for cooling an external surface of a mammalian body, the formulation comprising:
a) purified water at a concentration of about 67% to about 97% by volume;
b) denatured alcohol at a concentration of about 3.2% to about 26.7% by volume;
c) Aloe vera at a concentration of about 0.3% to about 4.2% by volume;
d)menthol at a concentration of about 0.3% to about 4.2% by volume; and at least one scented essential oil at a concentration of about 0.3% to about 3.3% by volume.

2. An absorbent cloth that when applied to an external surface of a mammalian body results in cooling of the external surface, the absorbent cloth comprising an absorbent cloth impregnated with a formulation comprising: purified water; denatured alcohol; Aloe vera; menthol; and at least one scented essential oil,
wherein the absorbent cloth is sealed in a package prior to application to the external surface of the mammalian body and when removed from the package, the absorbent cloth becomes cool within several seconds and remains cool for at least one hour without requiring refrigeration or hydration.

3. The absorbent cloth of claim 2, wherein the purified water is at a concentration of about 67% to about 97% by volume, the denatured alcohol is at a concentration of about 3.2% to about 26.7% by volume, the Aloe vera is at a concentration of about 0.3% to about 4.2% by volume, the menthol is at a concentration of about 0.3% to about 4.2% by volume, and the essential oil is at a concentration of about 0.3% to about 3.3% by volume.

4. The absorbent cloth of claim 2, further comprising a second essential oil.

5. The absorbent cloth of claim 2, wherein the denatured alcohol is ethyl alcohol.

6. The absorbent cloth of claim 2, wherein the Aloe vera is Aloe vera extract.

7. The absorbent cloth of claim 2, wherein the scented essential oil is citrus essential oil.

8. The absorbent cloth of claim 2, wherein the absorbent cloth is terry cloth.

9. The absorbent cloth of claim 2, wherein the absorbent cloth has a width in the range of about 6 inches to about 15 inches and a height in the range of about 9 inches to about 30 inches.

10. The absorbent cloth of claim 9, wherein the width is about 12 inches and the height is about 27 inches.

11. The absorbent cloth of claim 9, wherein the width is about 12 inches and the height is about 12 inches.

12. The absorbent cloth of claim 2, wherein the absorbent cloth is capable of cooling the external surface for a time period up to about four hours.

13. A method of cooling at least one external surface of a mammalian body, the method comprising the steps of:
(a) providing an absorbent cloth impregnated with a formulation comprising purified water, denatured alcohol, Aloe vera, menthol, and at least one scented essential oil; and
(b) applying the absorbent cloth to the at least one external surface, wherein when applied to the at least one external surface, the absorbent cloth results in cooling of the at least one external surface.

14. The method of claim 13, wherein the formulation further comprises a second essential oil.

15. The method of claim 13, wherein the denatured alcohol is ethyl alcohol.

16. The method of claim 13, wherein the Aloe vera is Aloe vera extract.

17. The method of claim 13, wherein the scented essential oil is citrus essential oil.

18. The method of claim 13, wherein the absorbent cloth is terry cloth.

19. The method cloth of claim 13, wherein the absorbent cloth has a width in the range of about 6 inches to about 15 inches and a height in the range of about 9 inches to about 30 inches.

20. The method of claim 19, wherein the width is about 12 inches and the height is about 27 inches.

21. The method of claim 19, wherein the width is about 12 inches and the height is about 12 inches.

22. The method of claim 13, wherein the cooling of the at least one external surface occurs for a time period between one and four hours.
